(19) United States
(12) Patent Application Publication
(10) Pub. No.: US 2023/0149604 A1
(43) Pub. Date: May 18, 2023

(54) POLYPEPTIDE MONOLAYER WITH HIGH POTENTIAL AND SUPER-HYDROPHILICITY, AND PREPARATION METHOD AND APPLICATION THEREOF

(71) Applicant: QILU UNIVERSITY OF TECHNOLOGY, Jinan (CN)

(72) Inventors: Jing XU, Jinan (CN); Zhen ZHANG, Jinan (CN); Tianduo LI, Jinan (CN); Huijun MA, Jinan (CN); Qing BAN, Jinan (CN)

(21) Appl. No.: 17/753,423

(22) PCT Filed: Nov. 25, 2020
(86) PCT No.: PCT/CN2020/131566

§ 371 Date: Mar. 2, 2022

(30) Foreign Application Priority Data
Jul. 30, 2020 (CN) 202010753455.6

(51) Int. Cl. A61L 31/04; A61L 31/10; A61L 31/12

(52) U.S. Cl. CPC A61L 31/044; A61L 31/10; A61L 31/129

(57) ABSTRACT

A polypeptide monolayer with a high surface potential and super-hydrophilicity, and a preparation method and application thereof. The polypeptide is composed of polypeptide molecules with a molecular weight of (1.48±0.2)×10⁵ g/mol, a height of the monolayer is 13.8-14.9 nm, the exposure of primary amino groups on the surface of the monolayer is 12-14%, a Zeta potential of the polypeptide monolayer is (−1)-5 mV; a contact angle of the monolayer is 10±1°. The monolayer serving as a surface coating material of a cardiovascular stent can be applied to treatment of cardiovascular diseases; and its super-hydrophilicity can allow a layer of hydration film to be formed on the surface of the material so as to effectively prevent protein adsorption.

### FIELD OF THE INVENTION

The present disclosure belongs to the field of natural macromolecules, relates to a polypeptide monolayer, and a preparation method and application thereof, and specifically relates to a polypeptide monolayer with a high surface potential and super-hydrophilicity, and a preparation method and application thereof.

### BACKGROUND OF THE INVENTION

Collagen polypeptide is a water-soluble protein obtained by chemically thermal degradation of collagen. It is one of the most commonly used biopolymers due to its excellent biocompatibility, plasticity, viscosity, richness, and low cost. As a biodegradable and renewable resource, collagen polypeptide is widely applied to the preparation of medical materials, biomimetic materials, and packing and coating materials. Immobilized bio-coatings are usually applied to the field of biomimetic stents to achieve an aim of loading biomolecules such as enzyme, lactose, and polydopamine, pharmaceutical molecules, synthetic macromolecules or small organic molecules, and a polypeptide monolayer prepared from collagen polypeptides has the advantage of easy and precise control of the loading amount.

However, a height of an immobilized bio-coating in the prior art is relatively great and difficult to control, generally greater than 100 nm. Furthermore, collagen polypeptide molecules contain a large number of polar groups such as amino groups, carboxy groups, and hydroxy groups, so that strong intermolecular hydrogen bonds are generated to form a network structure and then form a fragile thin film after dehydration. In addition, these groups and water molecules form hydrogen bonds to allow the polypeptide thin film to be susceptible to water absorption. These characteristics result in a fact that collagen polypeptide materials are fragile and soluble in water easily, which limits their application in some fields.

Secondary structures of a natural biological macromolecule can affect the exposure of functional groups in a polypeptide molecule so as to influence physicochemical properties of the surface of a monolayer, such as chemical properties, wettability, and electrical properties, and by modifying chemical properties, wettability, and electrical properties of the surface of a monolayer of an immobilized bio-coating, the immobilized bio-coating can be applied to the fields of preparation of cardiovascular and cerebrovascular stents, etc.

There are a lot of studies on regulation of a conformation of polypeptide molecules on an interface with a surfactant, but due to structural complexity of natural biological macromolecules, studies on chemical properties of the surface of a polypeptide monolayer are rarely reported, and application of polypeptide molecules is limited. In addition, strengthening studies on chemical properties of the surface of a polypeptide monolayer is beneficial to further modification of polypeptide molecules, which can further make up for its shortcomings.

### SUMMARY OF THE INVENTION

In order to solve the problems in the prior art, the present disclosure provides a polypeptide monolayer with a high surface potential and super-hydrophilicity, and a preparation method and application thereof. The present disclosure improves charges and hydrophilicity of the surface of a monolayer by modifying the exposure of primary amino groups on the surface of the polypeptide monolayer, so that the polypeptide monolayer of the present disclosure can be applied to the field of preparation of cardiovascular and cerebrovascular stents.

In the present disclosure, the exposure of primary amino groups is calculated by the following formula: the exposure of primary amino groups=molar weight of primary amino groups/collagen polypeptides (g).

In order to achieve the above objective, the present disclosure adopts the following technical solutions.

The present disclosure provides a polypeptide monolayer with a high surface potential and super-hydrophilicity, characterized in that the polypeptide is composed of polypeptide molecules with a molecular weight of (1.48±0.2)×10⁵ g/mol, a height of the monolayer is 13.8-14.9 nm, the exposure of primary amino groups on the surface of the monolayer is 12-14%, a Zeta potential of the polypeptide monolayer is (−1)-5 mV; and a contact angle of the monolayer is 10±1°.

Preferably, the polypeptide is collagen polypeptide. Preferably, the height of the monolayer is 14.2±0.1 nm.

Preferably, the polypeptide consists of 7.30±0.5% of glycine (Gly); 17.48±0.5% of valine (Vla); 36.97±0.5% of isoleucine (Ile); 13.85±0.5% of leucine (Leu); 2.68±0.5% of tyrosine (Tyr); 1.5±0.5% of phenylalanine (Phe); 4.41±0.5% of lysine (Lys); 0.45±0.5% of histidine (His); 3.45±0.5% of arginine (Arg); 5.96±0.5% of proline (Pro); and 5.95±0.5% of cysteine (Cys).

Preferably, secondary structures of the collagen polypeptide monolayer include 40-51% of α-helix; 10-15% of β-sheet; 2-7% of β-turn; and 31-42% of random coil.

Preferably, the polypeptide monolayer is composed of close-packed nanoparticles, and the spherical nanoparticles have an average particle size of 60±2 nm.

Preferably, the exposure of primary amino groups on the surface of the monolayer is 12.47±0.3% or 13.13±0.3%.

Preferably, the Zeta potential of the polypeptide monolayer is −(0.85±0.1) mV or 4.907±0.1 mV.

Preferably, the secondary structures of the monolayer include 50.98±0.2% of α-helix; 10.85±0.13% of β-sheet; 6.61±0.07% of β-turn; and 31.56±0.27% of random coil; or, 40.73±0.1% of α-helix; 14.97±0.13% of β-sheet; 2.55±0.08% of β-turn; and 41.75±0.22% of random coil. The content of the secondary structures of the above polypeptide monolayer is characterized by confocal Raman spectrometer.

The present disclosure also provides a composite film containing a polypeptide monolayer, including a polyethyleneimine thin film and a polypeptide monolayer, wherein the polyethyleneimine thin film and the polypeptide molecules are bound together via ionic bonds, a height of the polyethyleneimine thin film is 0.25-0.38 nm, and a height of the polypeptide monolayer is 13.8-14.9 nm.

The present disclosure also provides a preparation method of the above polypeptide monolayer, characterized by including the following steps:
(1) preparing a polypeptide solution at certain temperature, adding sodium dodecyl sulfate (SDS) serving as a surfactant to obtain a polypeptide-SDS mixed solution, and keeping the temperature of the mixed solution, wherein the concentration of sodium dodecyl sulfate in the mixed solution is 3.5-8.32 mmol/L;
(2) grinding and polishing the surface of a titanium sheet, immersing the titanium sheet in a mixed acid solution for treatment, rinsing until the titanium sheet is neutral, blow-drying with nitrogen, and further oven-drying;
(3) immersing the oven-dried titanium sheet in an aqueous solution of polyethyleneimine (PEI) for treatment, rinsing with water, blow-drying with nitrogen, and oven-drying to obtain a positively ionized titanium sheet deposited with PEI; and
(4) immersing the positively ionized titanium sheet in the polypeptide-SDS mixed solution obtained at step (1), depositing for 8-12 min, pulling the titanium sheet 20-25 times in deionized water, and blow-drying with high-purity nitrogen to obtain a polypeptide monolayer.

Preferably, the temperature at step (1) and the temperature during deposition at step (4) are both 50° C.

Preferably, at step (1), a concentration of the collagen polypeptide solution is 4 wt %; and the concentration of sodium dodecyl sulfate in the mixed solution is 3.5 mmol/L or 8.32 mmol/L.

Preferably, at step (1), a preparation method of the collagen polypeptide solution includes the following steps: mixing collagen polypeptides with deionized water, swelling at room temperature for 0.5 h, heating to 50° C., stirring for 2 h until the collagen polypeptides are completely dissolved; and regulating the pH to 10.00±0.02.

Preferably, at step (2), after being ground and polished by using metallographical sandpaper, the titanium sheet is ultrasonically washed with deionized water, absolute ethanol, and acetone for 15 min for each time, blow-dried with high-purity nitrogen, and dried in an oven at 60° C. for 12 h. Further preferably, a grinding and polishing method includes the following steps: grinding and polishing by using metallographical sandpaper to 800, 1,500, 3,000, 5,000, and 7,000 meshes in sequence.

Preferably, at step (2), the mixed acid solution is a mixed solution of 30% H₂O₂ and 98% H₂SO₄ in a volume ratio of 1:1, and the treatment time is 1 h.

Preferably, at step (3), the titanium sheet is treated in the aqueous solution of PEI for 20-40 min.

In the present disclosure, collagen polypeptide with a regular structure is obtained by dialyzing a commercially available polypeptide product.

The present disclosure also provides application of the above polypeptide monolayer serving as a surface coating material of a cardiovascular stent in treatment of cardiovascular diseases.

The relatively high exposure of primary amino groups on the surface of the polypeptide monolayer of the present disclosure will effectively increase the loading amount of cardiovascular drugs. The high surface potential can improve biocompatibility and hemocompatibility; and the surface high potential can improve cell adhesion, proliferation and differentiation abilities.

In addition, super-hydrophilicity of the monolayer allows a layer of hydration shell to be formed on the surface to prevent protein adsorption. When applied to cardiovascular stent materials, the monolayer can effectively prevent adsorption of common proteins such as fibrin and bovine serum albumin to avoid cardiovascular reocclusion.

The present disclosure has the following beneficial effects.

In the present disclosure, polypeptides are immobilized to the surface of a positively ionized substrate by an electrostatic self-assembly technology to prepare a polypeptide monolayer, and a Zeta potential and hydrophilicity of the surface of the monolayer are regulated by modifying the exposure of primary amino groups on the surface of the monolayer, which significantly improves cell attachment and proliferation and is beneficial to cell viability, so that the monolayer can be applied to the field of biomimetic stents.

The polypeptide monolayer of the present disclosure has super-hydrophilicity, which allows a layer of hydration shell to be formed on the surface of a material to effectively prevent protein adsorption so as to avoid cardiovascular reocclusion; and the high exposure of primary amino groups will effectively increase the loading amount of cardiovascular drugs.

### BRIEF DESCRIPTION OF THE DRAWINGS

FIG. 1 shows the effect of polypeptide concentration on ellipticity;

FIG. 2 shows an AFM image of a collagen polypeptide monolayer prepared from collagen polypeptides at a concentration of 4%;

FIG. 3 shows fluorescence intensities corresponding to different numbers of pulling;

FIG. 4A shows a height-distance curve chart of a polypeptide monolayer G-SDS_{6%};

FIG. 4B shows an AFM image of G-SDS_{6%};

FIG. 5A shows high-resolution N is XPS spectra (a: G-SDS_{6%}, b: G-SDS_{cmc}, c: G-SDS_{cac}, and d: 4% polypeptide monolayer);

FIG. 5B shows primary amino group contents of polypeptide monolayer;

FIG. 6 shows Zeta potentials and water contact angles of collagen polypeptide monolayers;

FIG. 7A shows contact angles of polypeptide monolayers (SDS_{cac});

FIG. 7B shows contact angles of polypeptide monolayers (SDS_{6%};

FIG. 7C shows contact angles of polypeptide monolayers (SDS_{cmc});

FIG. 8A shows ¹H NMR spectra of a product tetraphenylethylene-isothiocyanate (TPE-ITC) (TPE-CH₃);

FIG. 8B shows ¹H NMR spectra of a product tetraphenylethylene-isothiocyanate (TPE-ITC) (TPE-N₃);

FIG. 8C shows ¹H NMR spectra of a product tetraphenylethylene-isothiocyanate (TPE-ITC) (TPE-ITC);

FIG. 9A shows CLSM images of different samples (positively ionized titanium sheet);

FIG. 9B shows CLSM images of different samples (4% polypeptide-TPE);

FIG. 9C shows CLSM images of different samples (4% polypeptide);

FIG. 9D shows CLSM images of different samples (G-SDS_{cac}-TPE);

FIG. 9E shows CLSM images of different samples (G-SDS_{cac};

FIG. 9F shows CLSM images of different samples (G-SDS_{6%}-TPE);

FIG. 9G shows CLSM images of different samples (G-SDS_{6%});

FIG. 10 shows results of CCK-8 assays of the polypeptide monolayer G-SDS_{6%};

FIG. 11 shows results of MTT assays of the polypeptide monolayer G-SDS_{6%};

FIG. 12A shows cell viabilities of cells after being cloned with different samples (control group);

FIG. 12B shows cell viabilities of cells after being cloned with different samples (G-SDS_{cac %});

FIG. 12C shows cell viabilities of cells after being cloned with different samples (G-SDS_{6%});

FIG. 12D shows cell viabilities of cells after being cloned with different samples (cell viabilities of various cell treatment groups);

FIG. 13A shows fluorescence microscope images of collagen polypeptide monolayers before and after immersion for 7 d (4% polypeptide monolayer);

FIG. 13B shows fluorescence microscope images of collagen polypeptide monolayers before and after immersion for 7 d (4% polypeptide monolayer);

FIG. 13C shows fluorescence microscope images of collagen polypeptide monolayers before and after immersion for 7 d (G-SDS_{cmc});

FIG. 13D shows fluorescence microscope images of collagen polypeptide monolayers before and after immersion for 7 d (G-SDS_{cmc});

FIG. 13E shows fluorescence microscope images of collagen polypeptide monolayers before and after immersion for 7 d (G-SDS_{6%});

FIG. 13F shows fluorescence microscope images of collagen polypeptide monolayers before and after immersion for 7 d (G-SDS_{6%}); and

FIG. 13G shows fluorescence microscope images of collagen polypeptide monolayers before and after immersion for 7 d (G-SDS_{6%}).

### DETAILED DESCRIPTION OF THE EMBODIMENTS

The technical solutions of the present disclosure will be further described with reference to specific embodiments, collagen polypeptides used in the embodiments of the present disclosure are commercially available polypeptide products (A.R.) with a molecular weight of 5.00×10⁴-1.80×10⁵ g/mol, and polypeptides with a molecular weight of (1.48±0.2)×10⁵ g/mol is obtained by dialyzing the collagen polypeptides. Unless otherwise specified, other reagents are all commercially available.

Collagen polypeptide is an amphoteric polyelectrolyte, which can agglomerate into a spherical particle at the isoelectric point. Based on the aggregation behavior of collagen polypeptide, collagen polypeptides with a lower molecular weight can pass through a semi-permeable membrane by adjusting factors such as temperature, concentration, pH, and ionic strength, so as to achieve the purpose of separating from collagen polypeptides with a higher molecular weight. Study results obtained through gel electrophoresis and a laser particle analyzer show that collagen polypeptides with a narrow molecular weight distribution can be prepared by using dialysis tubing with a molecular-weight cutoff of 50,000 kDa under the conditions that the dialysis concentration of collagen polypeptides is 2%, the dialysis temperature is 45° C., and the concentration of NaCl is 0.9 mol·L⁻¹.

Comparison of CP, CA, M_{W}, and the isoelectric point (IP) of collagen polypeptides before and after dialysis is shown in Table 1, and comparison of amino acid types before and after dialysis is shown in Table 2. Determination results obtained through GPC show that a weight-average molecular weight M_{W} of the dialyzed collagen polypeptides is 1.48×10⁵ g·mol⁻¹, and M_{W}/Mₙ=1.43. Determination results obtained by the Kjeldahl method show that the protein content (CP) in the collagen polypeptides is 83.38%, and the amino acid content (CA) is 4.95×10⁻⁴ mol·g⁻¹, and determination results obtained by a primary amino group quantometer at 50° C. show that the primary amino group content in the dialyzed collagen polypeptide molecules is 4.95×10⁻⁴ g·mol⁻¹, and the molecular structure of the collagen polypeptides has no obvious change before and after dialysis. The collagen polypeptides are prepared into a 5% aqueous solution with a conductivity of 5.98 μS cm⁻¹, a conductivity of deionized water is 2.06 μS cm⁻¹, and the above results indicate that collagen polypeptides with a low molecular weight and inorganic salt mixed in the collagen polypeptides are dialyzed out.

TABLE 1

| | | | | IP, |
|---|---|---|---|---|
| Sample | CP, % | CA, mol g⁻¹ | Mw, GPC, g mol⁻¹ | Fluorescence |
| gelatin | 81.98 | 5.57 × 10⁻⁴ | 5.00 × 10⁴-1.80 × 10⁵ | 8.51 |
| dialyzed | 83.38 | 4.95 × 10⁻⁴ | (1.48 ± 0.2) × 10⁵ | 8.53 |
| gelatin | | | | |

TABLE 2

| Dialyzed- | | | | | |
|---|---|---|---|---|---|
| Gelatin | Conc/nmol | Conc/ng | Gelatin | Conc/nmol | Conc/ng |
| Gly | 249.42 | 5.33 | Gly | 205.12 | 5.35 |
| Val | 594.74 | 12.77 | Val | 460.81 | 12.00 |
| Ile | 1256.65 | 27.00 | Ile | 1036.75 | 27.03 |
| Leu | 472.25 | 10.14 | Leu | 387.73 | 10.10 |
| Tyr | 90.85 | 1.96 | Tyr | 69.08 | 1.80 |
| Phe | 50.79 | 1.09 | Phe | 39.10 | 1.01 |
| Lys | 150.35 | 3.22 | Lys | 125.13 | 3.26 |
| His | 15.41 | 0.32 | His | 15.54 | 0.39 |
| Arg | 117.38 | 2.52 | Arg | 101.92 | 2.66 |
| Pro | 202.42 | 4.35 | Pro | 169.95 | 4.43 |
| Cys | 202.78 | 4.34 | Cys | 165.99 | 4.33 |

### Example 1

### A Preparation Method of a Polypeptide Monolayer Included the Following Steps

(1) 50 mL of 4 wt % collagen polypeptide solution was prepared: 100 mL of collagen polypeptide was precisely weighed and placed into a three-neck flask, deionized water was precisely weighed and poured into the three-neck flask, the collagen polypeptides swelled at room temperature for 0.5 h, the three-neck flask was placed into a water bath at 50±1° C., the solution was heated and stirred for 2 h until the collagen polypeptides were completely dissolved, the pH of the solution was regulated with 2 mol/L sodium hydroxide to 10.00±0.02, and the solution was stabilized in the water bath for 0.5 h.
(2) SDS serving as a surfactant was added to the above collagen polypeptide solution to obtain a collagen polypeptide-SDS mixed solution in which the concentration (CAC, namely the critical aggregation concentration of SDS at 50° C.) of SDS was 3.50 mmol/L; and the mixed solution was stabilized in the water bath for δ h for later use.
(3) A rectangle titanium sheet with a size of 1 cm×1 cm×1 mm was cut, ground and polished by using metallographical sandpaper to 800, 1,500, 3,000, 5,000, and 7,000 meshes in sequence, ultrasonically washed with deionized water, absolute ethanol, and acetone in sequence for 15 min for each time, blow-dried with high-purity nitrogen, and dried in an oven at 60° C. for 12 h for later use. A mixed acid solution of 30% H₂O₂ and 98% H₂SO₄ in a volume ratio of 1:1 was prepared and cooled to room temperature, the above treated titanium sheet was treated with the mixed acid solution for 1 h, rinsed with tap water until the titanium sheet was neutral, washed 5 times with deionized water, blow-dried with high-purity nitrogen, and dried in the oven at 60° C. for 12 h for later use.
(4) A 1 mg/mL aqueous solution of polyethyleneimine (PEI) was prepared, the above acid-etched titanium sheet was treated with the PEI solution at room temperature for 0.5 h, washed 5 times with deionized water to remove loosely bound charges, blow-dried with high-purity nitrogen, and dried in the oven at 60° C. for 12 h for later use. The positively ionized titanium sheet was placed into a deposition box, and the above prepared polypeptide solutions of different systems were poured into the deposition box respectively, the titanium sheet was subjected to deposition at 50° C. for 10 min, and pulled 20 times in deionized water, blow-dried with high-purity nitrogen, and stored in nitrogen.

The obtained polypeptide monolayer was denoted as G-SDS_{cac}.

### Example 2

### A Preparation Method of a Polypeptide Monolayer Included the Following Steps

(1) 50 mL of 4 wt % collagen polypeptide solution was prepared: 100 mL of collagen polypeptide was precisely weighed and placed into a three-neck flask, deionized water was precisely weighed and poured into the three-neck flask, the collagen polypeptides swelled at room temperature for 0.5 h, the three-neck flask was placed into a water bath at 50±1° C., the solution was heated and stirred for 2 h until the collagen polypeptides were completely dissolved, the pH of the solution was regulated with 2 mol/L sodium hydroxide to 10.00±0.02, and the solution was stabilized in the water bath for 0.5 h.
(2) SDS serving as a surfactant was added to the above collagen polypeptide solution to obtain a collagen polypeptide-SDS mixed solution in which the concentration of SDS was 8.32 mmol/L (6 wt %); and the mixed solution was stabilized in the water bath for δ h for later use.
(3) A rectangle titanium sheet with a size of 1 cm×1 cm×1 mm was cut, ground and polished by using metallographical sandpaper to 800, 1,500, 3,000, 5,000, and 7,000 meshes in sequence, ultrasonically washed with deionized water, absolute ethanol, and acetone in sequence for 15 min for each time, blow-dried with high-purity nitrogen, and dried in an oven at 60° C. for 12 h for later use. A mixed acid solution of 30% H₂O₂ and 98% H₂SO₄ in a volume ratio of 1:1 was prepared and cooled to room temperature, the above treated titanium sheet was treated with the mixed acid solution for 1 h, rinsed with tap water until the titanium sheet was neutral, washed 5 times with deionized water, blow-dried with high-purity nitrogen, and dried in the oven at 60° C. for 12 h for later use.
(4) A 1 mg/mL aqueous solution of polyethyleneimine (PEI) was prepared, the above acid-etched titanium sheet was treated with the PEI solution at room temperature for 0.5 h, washed 5 times with deionized water to remove loosely bound charges, blow-dried with high-purity nitrogen, and dried in the oven at 60° C. for 12 h for later use. The positively ionized titanium sheet was placed into a deposition box, and the above prepared polypeptide solutions of different systems were poured into the deposition box respectively, the titanium sheet was subjected to deposition at 50° C. for 10 min, and pulled 20 times in deionized water, blow-dried with high-purity nitrogen, and stored in nitrogen.

The obtained polypeptide monolayer was denoted as G-SDS_{6%}.

### Comparative Example 1

Collagen polypeptide solutions at different concentrations of 1-5 wt % were prepared: the mass of collagen polypeptides and the volume of deionized water required were calculated, collagen polypeptides were precisely weighed and placed into a 50 mL three-neck flask, deionized water was precisely weighed and poured into the three-neck flask, the polypeptides swelled at room temperature for 0.5 h, the three-neck flask was placed into a water bath at 50° C., the solution was stirred for 2 h until the collagen polypeptides were completely dissolved, and the pH of the solution was regulated with 1 mol/L sodium hydroxide to 10.00±0.02 for later use.

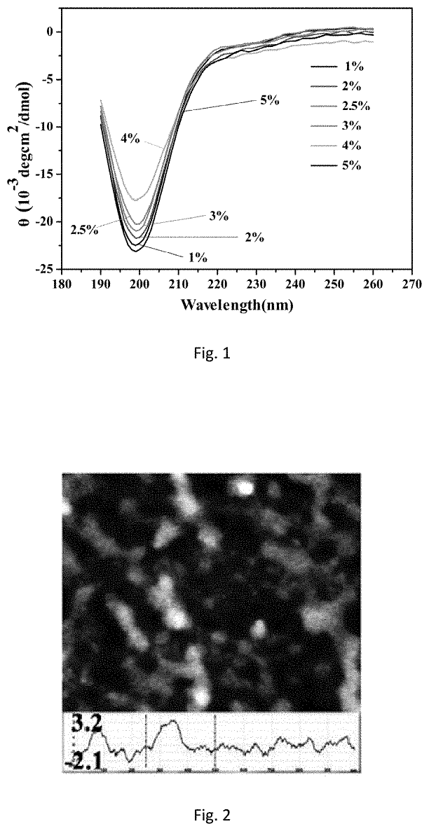

The above collagen polypeptide solutions at different concentrations were characterized by circular dichroism chromatography (CD), and the size of circular dichroism is usually determined based on a molar extinction coefficient difference Δε (M⁻¹·cm⁻¹); a molar ellipticity 0. CD detection was carried out on a Chirascan system (Applied Photophysics Ltd., UK), and the blowing rate of nitrogen was 35 mL/min. Concentrations of proteins in all the solutions were reduced to 0.16 mg/mL by dilution, the mixed sample was balanced at 50° C. for 1 h, and meanwhile, 200 μL of solution was taken and detected in a 1 mm sample pool at 50° C., and the temperature during detection was kept at 50° C. Spectra within a range of 190-260 nm were recorded, the resolution was 0.2 nm, and the samples were scanned δ times. Data processing: the spectrum of the buffer solution was subtracted to correct the baseline, the CD spectra were normalized in units of molar ellipticity, and the content of secondary structures was calculated by the peak regression calculation method and the CONTIN fitting program. The effect of polypeptide concentration on secondary structures of polypeptide is shown in FIG. 1 and Table 3.

TABLE 3

| Concentration | | | | | |
|---|---|---|---|---|---|
| (wt) | α-helix | Antiparallel | parallel | β-turn | random coil |
| 1% | 5% | 11.5% | 2% | 32.5% | 50.1% |
| 2% | 5.2% | 11.9% | 2% | 32% | 49.5% |
| 2.5% | 5.2% | 13.6% | 2.1% | 30.8% | 47.7% |
| 3% | 5.1% | 13.5% | 2.1% | 31% | 48% |
| 4% | 5.6% | 16.8% | 2.3% | 28.4% | 44.7% |
| 5% | 5.1% | 10.4% | 2.1% | 30.8% | 51.2% |

As shown in Table 3 and FIG. 1, α-helix, Antiparallel β-sheet, and parallel β-sheet structures show a trend of increasing first and then decreasing as the mass concentration of the polypeptide increases from 1% to 5%, and reach the maximum at the concentration of 4%; and β-turn and random coil structures show a trend of decreasing first and then increasing, and reach the minimum at the concentration of 4%. These results indicate that the secondary structures of the polypeptide molecule change greatly at the concentration of 4%. This concentration is just at the boundary between the contact concentration and the entanglement concentration of polypeptide molecules. Therefore, in the present disclosure, when a polypeptide monolayer is prepared, the mass concentration of polypeptide is preferably 4%.

### Comparative Example 2

The difference between a preparation method of a polypeptide monolayer of the present example and that of Example 1 was that no surfactant was added in the preparation process of the monolayer, only collagen polypeptides were deposited onto a positively ionized titanium sheet, and other conditions were the same as those of Example 1.

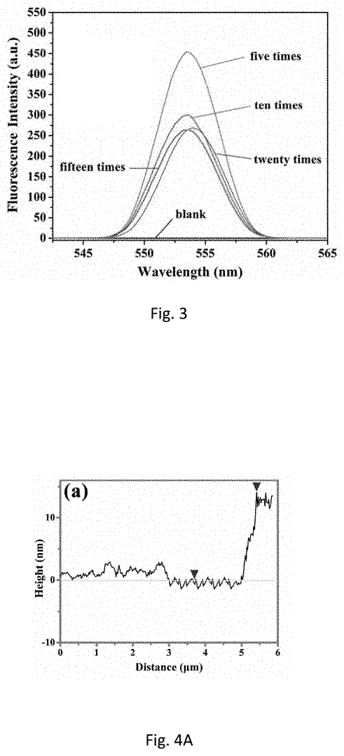

A polypeptide solution at a concentration of 4% was deposited onto a titanium sheet treated with PEI at 50° C. for 10 min, the titanium sheet was pulled 20 times, and polypeptide molecules were loosely arranged, as shown in FIG. 2. The obtained polypeptide monolayer was denoted as G.

### Comparative Example 3

### A Preparation Method of a Polypeptide Monolayer Included the Following Steps

(1) 50 mL of 4 wt % collagen polypeptide solution was prepared: 100 mL of collagen polypeptide was precisely weighed and placed into a three-neck flask, deionized water was precisely weighed and poured into the three-neck flask, the collagen polypeptides swelled at room temperature for 0.5 h, the three-neck flask was placed into a water bath at 50±1° C., the solution was heated and stirred for 2 h until the collagen polypeptides were completely dissolved, the pH of the solution was regulated with 2 mol/L sodium hydroxide to 10.00±0.02, and the solution was stabilized in the water bath for 0.5 h.
(2) SDS serving as a surfactant was added to the above collagen polypeptide solution to obtain a collagen polypeptide-SDS mixed solution in which the concentration (CMC, namely the critical micelle concentration of SDS at 50° C.) of SDS was 7.50 mmol/L; and the mixed solution was stabilized in the water bath for δ h for later use.
(3) A rectangle titanium sheet with a size of 1 cm×1 cm×1 mm was cut, ground and polished by using metallographical sandpaper to 800, 1,500, 3,000, 5,000, and 7,000 meshes in sequence, ultrasonically washed with deionized water, absolute ethanol, and acetone in sequence for 15 min for each time, blow-dried with high-purity nitrogen, and dried in an oven at 60° C. for 12 h for later use. A mixed acid solution of 30% H₂O₂ and 98% H₂SO₄ in a volume ratio of 1:1 was prepared and cooled to room temperature, the above treated titanium sheet was treated with the mixed acid solution for 1 h, rinsed with tap water until the titanium sheet was neutral, washed 5 times with deionized water, blow-dried with high-purity nitrogen, and dried in the oven at 60° C. for 12 h for later use.
(4) A 1 mg/mL aqueous solution of polyethyleneimine (PEI) was prepared, the above acid-etched titanium sheet was treated with the PEI solution at room temperature for 0.5 h, washed 5 times with deionized water to remove loosely bound charges, blow-dried with high-purity nitrogen, and dried in the oven at 60° C. for 12 h for later use. The positively ionized titanium sheet was placed into a deposition box, and the above prepared polypeptide solutions of different systems were poured into the deposition box respectively, the titanium sheet was subjected to deposition at 50° C. for 10 min, and pulled 20 times in deionized water, blow-dried with high-purity nitrogen, and stored in nitrogen.

The obtained polypeptide monolayer was denoted as G-SDS_{cmc}.
1. Determination of Heights of the Polypeptide Monolayers

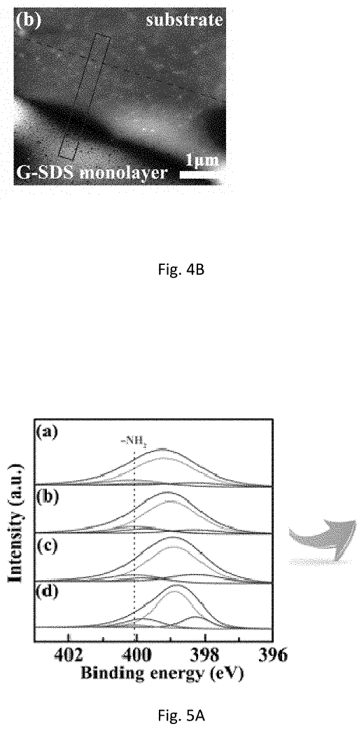

After the PEI-treated titanium sheet was deposited with collagen polypeptides,−COO⁻in a polypeptide molecule and —NH₃⁺in PEI could form a strong ionic bond. In order to verify that the collagen polypeptide molecules are bound to a substrate via ionic bonds rather than physical adsorption, fluorescence intensities corresponding to different numbers of pulling in the deposition process of the polypeptide monolayer were determined. As the number of pulling is increased (5-20 times), the polypeptides that are physically adsorbed onto the substrate are washed away while those bound via ionic bonds are firmly immobilized onto the substrate. It can be seen from FIG. 3 that after the titanium sheet is pulled 15 times, the fluorescence intensity is no longer decreased, which indicates that the collagen polypeptides physically adsorbed onto the substrate are removed.

In the present disclosure, the surface morphology of the monolayer was detected by using a Multimode 8 AFM (Bruker, Germany). The polypeptide monolayer sample was placed onto a working table, and the morphology of the sample was detected in a Peak Force mode. Determination of the height of the monolayer: when a monolayer was prepared by a deposition method, half of a titanium sheet was wrapped with tin foil to keep it from being contaminated by the solution. During detection, a boundary of the titanium sheet was found by using a build-in auxiliary optical system of the atomic force microscope, a detection range was set to 20 μm to span the substrate and the sample area, the sample was scanned by an AFM tip along the boundary from the height corresponding to the monolayer substrate to the bottom of the boundary, and 3 different areas were scanned so as to obtain an average height of the monolayer. The scanning speed was 0.977 Hz, the scanning ranges were 20μm, 10μm, 5 μm, and 1 μm, respectively, and the data processing software was build-in NanoScope Analysis of AFM.

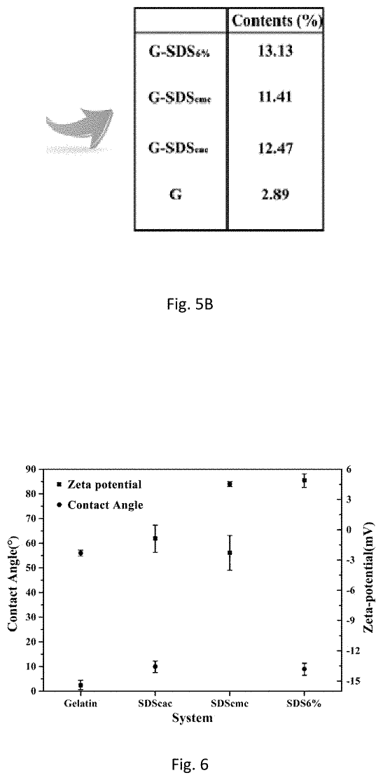

It can be found from an AFM image in FIG. 4 that an average height of the polypeptide monolayer (G-SDS_{6%}) obtained in Example 2 is about 14.2 nm. In addition, the collagen polypeptide monolayers obtained in Examples 1 and 2 are both composed of close-packed nanoparticles, and spherical nanoparticles have an average particle size of about 60 nm.
2. Determination of the Exposure of Primary Amino Groups on the Surface of the Polypeptide Monolayer

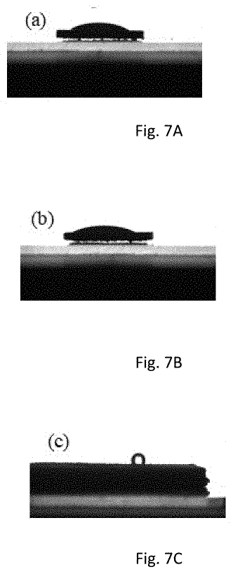

The samples obtained in Examples 1 and 2, and Comparative Examples 2 and 3 were characterized by XPS, and N elements were subjected to peak separation. The binding energy for primary amines is 400.05 eV, the binding energy for amido bonds is 398.89 eV, and the binding energy for secondary amines is 398.26 eV. The XPS data can also be used to determine changes in the binding energy and local chemical state so as to achieve semiquantitative analysis of functional groups. High-resolution spectra of N is core regions (from 396 to 402 eV) and the exposure of primary amino groups are shown in FIG. 5. The exposure of primary amino groups in the polypeptide monolayer (G-SDS_{6%}) is 13.13%, the exposure of primary amino groups in the polypeptide monolayer (G-SDS_{cac}) is 12.47%, while the exposure of primary amino groups in the polypeptide monolayer (G-SDS_{cmc}) is 11.41%, and the exposure of primary amino groups in the polypeptide monolayer (G) is only 2.89%. Peaks in the N is high-resolution spectra were separated by using CasaXPS and the primary amino group content was calculated, and XPS and Raman results show that the exposure of primary amino groups in the collagen polypeptide monolayer is related to the increased β-sheet and random coil structures, and is also related to the non-covalent interaction between the collagen polypeptides and surfactants at different concentrations.
3. Determination of Wettability and Charge Properties of the Surface of the Monolayer

A water contact angle (CA) of the monolayer sample was determined at room temperature by using a DSA-100 optical contact angle measuring system (KRUSS, Germany). 2 mL of deionized water was dropwise added to the sample by using an automatic assign controller, and CA was automatically determined by the Laplace-Young fitting algorithm. Five different positions on the sample were determined to obtain an average value of CA, and photos were taken by using a digital camera (SONY, Japan). A Zeta potential of the surface of the monolayer was determined by using a SurPASS electrokinetic solid surface analyzer.

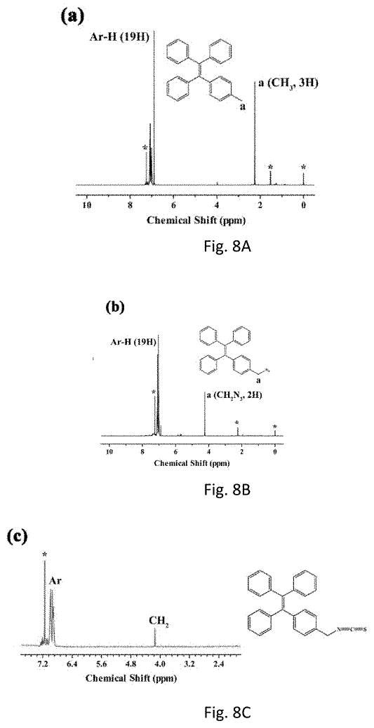

1 mM Na₂SO₄ solution was used as an electrolyte to determine the Zeta potential of the surface of the monolayer. FIG. 6 shows Zeta potentials of the surfaces of the collagen polypeptide monolayers containing SDS_{cmc} The numerical order of the surface Zeta potentials is that: 4 wt % polypeptide monolayer <G-SDS_{cmc}<G-SDS_{cac}<G-SDS_{6%}. Results show that a higher Zeta potential can be detected when the concentration of SDS is δ wt % or CAC. The comparison of the results and the XPS analysis results indicates that increase in the Zeta potential is related to increase in content of primary amino groups. The exposure of amino groups on the surface promotes the positive charge properties. A Zeta potential of the 4 wt % polypeptide monolayer is −15.6 mV; a Zeta potential of the G-SDS_{cmc} monolayer is −2.29 mV; a Zeta potential of the G-SDS_{cac} polypeptide monolayer is −0.85 mV; and a Zeta potential of the G-SDS_{6%} polypeptide monolayer is 4.907 mV. A high surface potential can improve cell adhesion, proliferation, and differentiation abilities.

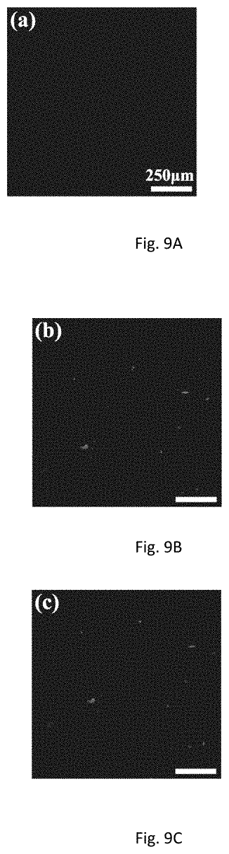

Wettability of the surface can be directly reflected by a water contact angle, as shown in FIG. 6. A pure Ti sheet shows hydrophobicity and has a contact angle of 101.4±0.2°, and a contact angle of the surface of the 4 wt % collagen polypeptide monolayer is 56.1±1.2°. A contact angle of the surface of G-SDS_{cmc} is-84°, while the surfaces of G-SDS_{cac} and G-SDS_{6%} are super-hydrophilic and have a contract angle of about 10°, as shown in FIG. 7. The results indicate that the wettability is related to the exposure of primary amino groups and the structure of the monolayer. Due to the super-hydrophilicity, a layer of hydration shell is formed on the surface to avoid protein adsorption. By use of its super-hydrophilicity, the surface coating material is applied to cardiovascular stents to prevent protein adsorption and avoid cardiovascular reocclusion.
4. Calculation of Content of Secondary Structures of Polypeptides in the Polypeptide Monolayer

In the vibration process of the amide groups, Raman peaks of amide I and amide III bands are very sensitive to conformational changes of protein backbone. In amide III band, four secondary structures, i.e. α-helix, β-sheet, β-turn, and random coil, are located at 1265-1300 cm⁻¹, 1230-1240 cm⁻¹, 1305 cm⁻¹, and 1240-1260 cm⁻¹, respectively. SAMs of G-SDS mounted on the surface of Ti were characterized by Raman spectra, a Raman spectrum of amide III band reveals surface-sensitive information on secondary structures of the collagen polypeptide monolayer. Content of the secondary structures of the surface of the polypeptide monolayer was characterized by using a confocal Raman spectrometer, and a determination method included: a vibrational Raman spectrum of the sample was recorded by using a LabRAM HR800 Raman spectrometer (Horiba Jobin Yvon, France) equipped with a He—Ne laser (632.8 nm) and 600 groove mm⁻¹ grating. The measurement accuracy of Raman intensity was about 1.2 cm⁻¹. A Raman reference spectrum of the sample was obtained under the conditions of a laser power of 1.1 mw, an irradiation time of 1 s, and 30 accumulations. Raman spectra of the PEI-modified sample and the collagen polypeptide-covered sample were obtained under the conditions of a laser power of −0.06 mW, an irradiation time of 1 s, and 10 scans. In all Raman experiments, the orientation of a platform was carefully controlled to allow a polarizer to which a laser was input to be parallel to a bow-tie shaft. The spectra were processed on PeakFit of Systat software. A baseline was determined, and the position of each sub-peak was determined with reference to a deconvolution spectrum and a third derivative spectrum. It helps to resolve overlapping sub-peaks and distinguish interference from noise peaks. Percentage of the secondary structures was obtained by the curve-fitting method. Then, the peak height of each sub-peak, a peak width at half height, the Gaussian content were changed to minimize a root-mean-square of curve-fitting, and the root-mean-square of curve-fitting was characterized with the secondary peak area. Amide III band in the original spectrum was analyzed by the curve-fitting method. In the region of amide III band, typical absorption peaks of α-helix, β-sheet, β-turn, and random coil structures appear at 1265-1300 cm⁻¹, 1230-1240 cm⁻¹, 1305 cm⁻¹, and 1240-1260 cm⁻¹, respectively.

The content of the secondary structures of the surface of the polypeptide monolayer is shown in Table 4, and by adding SDS at different concentrations, the content of α-helix, β-sheet, β-turn, and random coil in the monolayer is changed. As the concentration of SDS is increased from CAC to δ wt %, the total content of α-helix and β-turn is reduced, while the total content of β-sheet and random coil is increased. In SDS_{cac}, the total content of α-helix and β-turn is about 60%. However, in SDS_{6%}, the total content of β-sheet and random coil is about 57%. In addition, the content of α-helix in the collagen polypeptide monolayer containing SDS is significantly increased.

TABLE 4

| | α-helix | β-sheet | β-turn | random | α-helix + | β-sheet + |
|---|---|---|---|---|---|---|
| | (%) | (%) | (%) | coil (%) | β-turn (%) | random coil |
| Gelatine | 31.76 ± 0.18 | 11.65 ± 0.09 | 1.80 ± 0.06 | 54.79 ± 0.29 | 33.56 ± 0.20 | 66.44 ± 0.15 |
| G-SDS_{cac} | 50.98 ± 0.26 | 10.85 ± 0.13 | 6.61 ± 0.07 | 31.56 ± 0.27 | 57.59 ± 0.23 | 42.41 ± 0.27 |
| G-SDS_{6%} | 40.73 ± 0.14 | 14.97 ± 0.13 | 2.55 ± 0.08 | 41.75 ± 0.22 | 43.28 ± 0.28 | 56.72 ± 0.19 |

5. Characterization of Primary Amino Group Distribution Points on the Surface of the Monolayer

Probe synthesis: a fluorescent probe molecule tetraphenylethylene-isothiocyanate (TPE-ITC) responsive to primary amino groups was synthesized to visually characterize the distribution of primary amino groups on the surface of the polypeptide monolayer. Specifically, the probe was 1-[4-(methyl isothiocyanate)phenyl]-1,2,2-triphenylethylene (TPE-ITC), which was an adduct of tetraphenylethylene (TPE) and isothiocyanate (ITC).

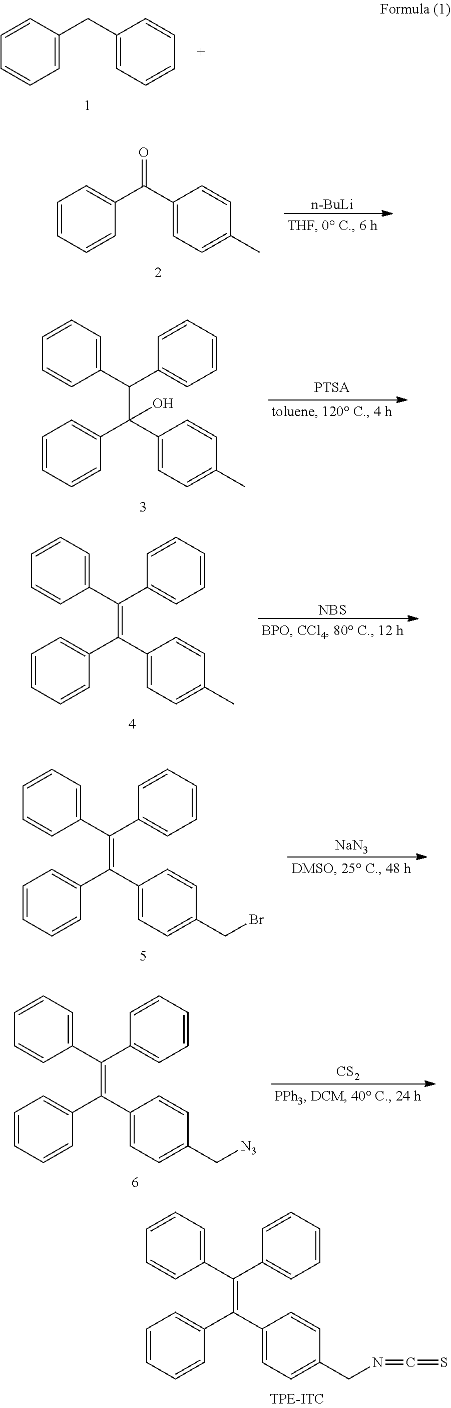

As shown in Formula (1) above, a synthesis method specifically included 5 steps. 0 In a 250 mL two-neck round-bottomed flask, 5.05 g (30 mmol) of diphenylmethane was dissolved in 100 mL of distilled tetrahydrofuran in the presence of N₂. After the mixture was cooled to 0° C., 15 mL of n-butyllithium (2.5 M hexane solution, 37.5 mmol) was slowly added by using a syringe. The mixture was stirred at 0° C. for 1 h. Then, 4.91 g (25 mmol) of 4-methylbenzophenone was added to the reaction mixture. The mixture was heated to room temperature and stirred for δ h. A compound 3 was synthesized.

\textcircled{2} The reaction mixture was quenched with a saturated ammonium chloride solution, and extracted with dichlorocarbene. An organic layer was collected and concentrated. The crude product and 0.20 g of p-toluenesulfonic acid were dissolved in 100 mL of toluene. The mixture was subjected to heating reflux for 4 h. After being cooled to room temperature, the reaction mixture was extracted with dichlorocarbene. An organic layer was collected and concentrated. The crude product was purified by silica gel column chromatography in which hexane was used as an eluent to obtain a white solid 4.

\textcircled{3} In a 250 mL round-bottomed flask, 5.20 g (15.0 mmol) of white solid 4, 2.94 g (16.0 mmol) of N-bromosuccinimide, and 0.036 g of benzoyl peroxide were subjected to reflux in 80 mL of carbon tetrachloride solution for 12 h. After the reaction was completed, the mixture was extracted with dichloromethane and water. Organic layers were combined and dried with anhydrous magnesium sulfate. The crude product was purified by silica gel column chromatography in which hexane was used as an eluent to obtain a white solid 5.

\textcircled{4} In a 250 mL two-neck round-bottomed flask, 1.70 g (4 mmol) of white solid 5 and 0.39 g (6 mmol) of sodium azide were dissolved in dimethyl sulfoxide in the presence of N2. The mixture was stirred at room temperature overnight (25° C., 48 h). Then, a large amount (100 mL) of water was added, and the solution was extracted 3 times with diethyl ether. Organic layers were combined and dried with anhydrous magnesium sulfate. The crude product was purified by silica gel column chromatography in which hexane/chloroform (v/v=3:1) was used as an eluent to obtain a white solid 6.

\textcircled{5} Tetraphenylethylene (the white solid 6, 0.330 g, 0.852 mmol) containing functionalized azide groups and triphenylphosphine (0.112 g, 0.426 mmol) were added into a two-neck flask, evacuated under vacuum, and washed 3 times with dry nitrogen. Carbon disulfide (0.55 g, 7.242 mmol) and distilled dichloromethane (50 mL) were added into the flask, and the mixture was stirred. The obtained reaction mixture was subjected to reflux overnight, and the solvent was removed under reduced pressure. The crude product was precipitated with cold diethyl ether (250 mL), and precipitates were filtered and washed 3 times. Finally, the product was dried under vacuum to obtain a white solid TPE-ITC.

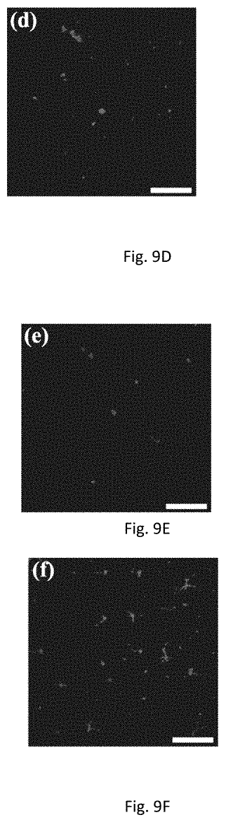

First, the synthetic product (tetraphenylethylene-isothiocyanate (TPE-ITC)) was characterized by H-nuclear magnetic resonance spectroscopy. The ¹H NMR of the product was obtained by using an AVANCE II 400 NMR spectrometer (Bruker, Germany). The sample to be detected with a size of −0.5 cm was placed in a nuclear magnetic resonance tube, then 0.6 mL of deuterated chloroform was added to dissolve it completely, tetramethylsilane (TMS) was used as the internal standard, and the sample was detected by manual shimming at room temperature, and the number of scans was 64, the obtained ¹H NMR spectrum was processed by using MestReNova software, and results are shown in FIG. 8. FIG. 8a: ¹H NMR (CDCl₃, 400 MHz), δ (TMS, ppm): 7.15-6.98 (m, 15H), 6.89 (s, 4H), 2.24 (s, 3H); FIG. 8b: ¹H NMR (CDCl₃, 400 MHz), δ (TMS, ppm): 7.12-6.90 (m, 19H), 4.24 (s, 2H); and FIG. 8c: ¹H NMR (400 MHz, CDCl₃) δ (ppm): 6.90-7.15 (m, 19H), 4.61 (s, 2H). For example, due to resonance of methylene groups of TPE and ITC, the product shows a peak at δ 4.16 in the ¹H NMR spectrum (FIG. 8c).

The above results indicate that a TPE-ITC molecular probe for imaging and functionalizing primary amino groups is synthesized, in which the reactive ITC group has a sensitive response to the primary amino groups. Therefore, TPE-ITC is a typical fluorescent molecule having an aggregation-induced emission (AIE) property. The AIE property of TPE-ITC enables a TPE-polypeptide bioconjugate to fluoresce strongly by attaching a large number of AIE labels to collagen polypeptide chains. The fluorescence output of the bioconjugate can be greatly enhanced (up to 2 orders of magnitude) by simply increasing its degree of labelling (DL). The AIE probe allows for real-time observation of primary amino groups.

The primary amino groups on the surface of the collagen polypeptide monolayer were labelled with the synthetic TPE-ITC, and the labelling procedure is shown in Formula (2).

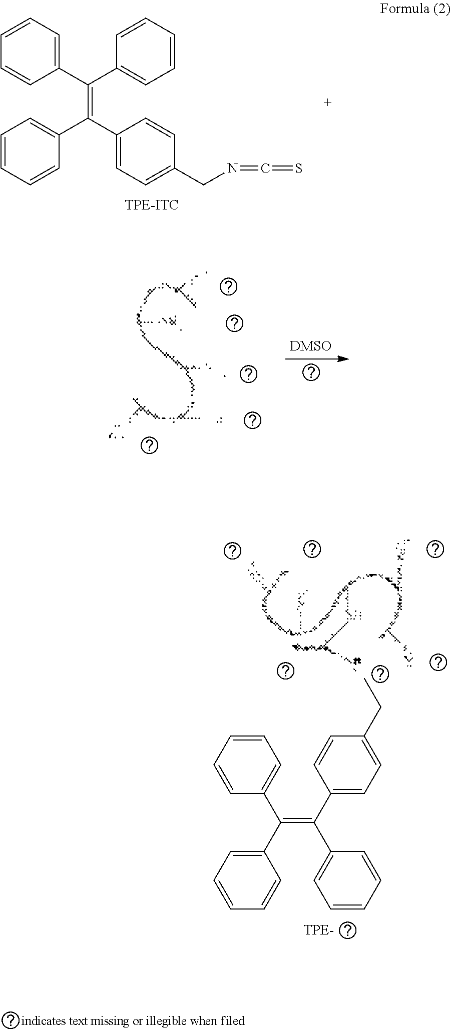

Specifically, the labelling steps were as follows: 0.8 mg/mL TPE-ITC/DMSO solution was prepared, 0.5 mL of solution was taken by using a 1 mL syringe, 9 drops were added to 5 mL of Na₂CO₃/NaHCO₃ buffer solution, and the mixed solution was subjected to ultrasonic treatment for 10 min until TPE-ITC was uniformly dispersed. The polypeptide monolayer was placed into a deposition box, the mixed solution subjected to ultrasonic treatment was slowly poured into the deposition box, the polypeptide monolayer was reacted at 50° C. for 2 h, and after the reaction was completed, the polypeptide monolayer was pulled 10 times in DMSO to remove unlabeled TPE-ITC, blow-dried with high-purity nitrogen, and stored in nitrogen.

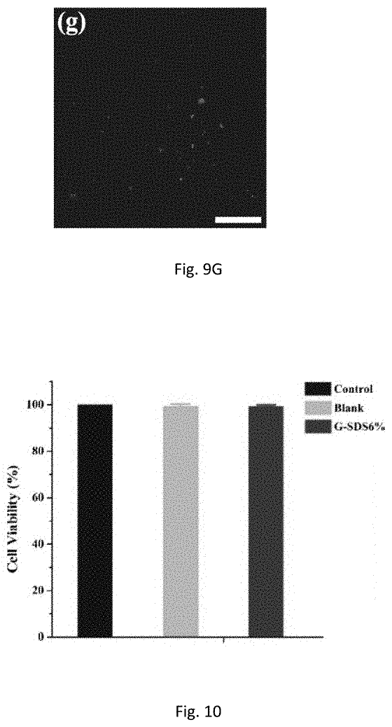

Confocal laser scanning microscopy (CLSM) images of the samples were obtained by using a TCS SP8 STED 3× confocal laser scanning microscope (Leica Camera AG, Germany) equipped with an argon-ion laser and two photomultiplier tubes. The resonance scanner was used together with an ultra-sensitive HyDTM detector. The samples were excited with a laser of 405 nm, and fluorescence was detected at 430-493 nm. The CLSM images are shown in FIG. 9, it can be seen that the content of primary amino groups in the G-SDS_{6%} monolayer (f in FIG. 9) is greater than that of the monolayer containing SDS at different concentration. The CLSM results are consistent with those of the XPS analysis. The collagen polypeptide molecule contains phenylalanine, tryptophan, and tyrosine, which can auto-fluoresce. In the experiment, the sample without TPE-ITC labelling was characterized by CLSM as a reference to verify that enhancement of fluorescence after labelling is caused by exposure of primary amino groups (c, e, and g in FIG. 9).
6. Test of Biocompatibility of the Monolayer

Cytocompatibility of the monolayer sample was tested by using cholecystokinin octapeptide (CCK-8) and methyl thiazolyl tetrazolium (MTT). A material to be tested was prepared in the same size as wells in a 12-well cell culture plate. The pure Ti and G-SDS_{6%} monolayer samples were placed into the wells, and three parallel wells were used for each sample. Human umbilical vein endothelial cells (HUVECs, 5×10⁵ cells/mL) were inoculated into each well and cultured in an RPMI 1640 medium containing 10% fetal bovine serum (FBS) at 37° C. and 5% CO₂ for 24 h. Then, the cells were washed twice with a serum-free minimum essential medium (MEM) Eagle, and 15 μL of CCK-8 solution was added to each well containing 100 μL of serum-free MEM. After the cells were incubated at 37° C. and 5% CO₂ for 1 h, 100 μL of mixture was transferred to another 12-well plate, as residual G-SDS_{6%} monolayer would affect absorbance at 450 nm. With absorbance at 655 nm as reference, the absorbance of the mixed solution at 450 nm was measured by using an iMark microplate reader, and the wells containing only the cells and the medium served as a control. The cell viability was calculated by the following formula:

Viability_{CCK-}8=Sample abs_{450-655 nm}/Positive control abs_{450-655 nm})×100

In addition to the CCK-8 assay, the cell viability of HUVECs was tested by an MTT assay. The cell viability was calculated by the following formula, and the cells incubated without the monolayer served as a control.

Viability_{MTT}=(Sample abs_{570-655 nm}/control abs_{570-655 nm})×100

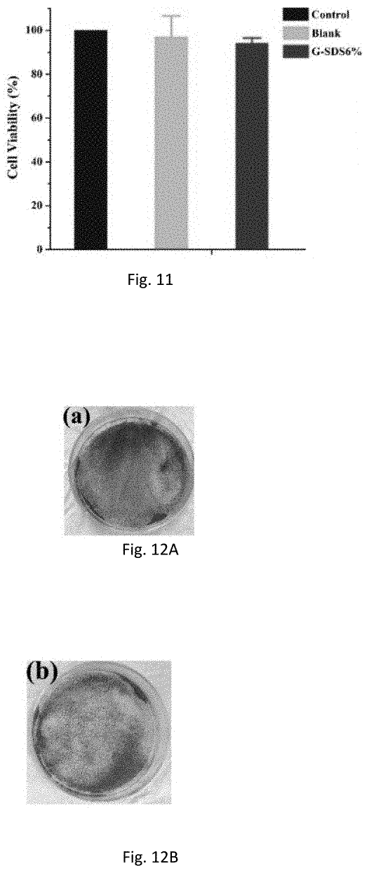

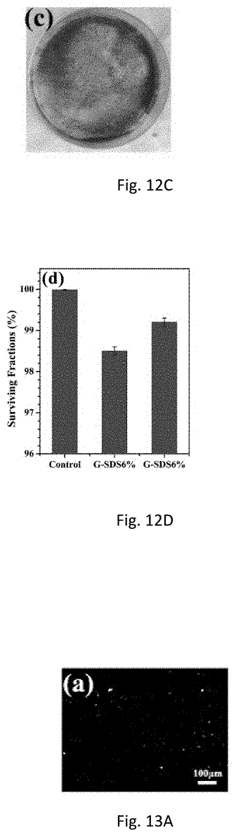

Results of the CCK-8 assay indicate that compared with the control group, G-SDS_{6%} serving as a modifying surface has no effect on cell viability and growth (FIG. 10). Results of the MTT assay also show that the G-SDS_{6%} monolayer is almost non-toxic to HUVECs (FIG. 11).

Cell cloning experiment: MCF-7 cells were cultured in a 60 mm culture dish, incubated in DMEM at 37° C. and 5% CO₂ for 24 h, and then the cells were treated differently: a blank control group and a G-SDS_{6%} monolayer group. 8 h later, the cells were washed 3 times with PBS (10 mM, pH=7.4). Then, the cells were cultured in fresh DMEM at 37° C. and 5% CO₂ for another 10 d, immobilized with 4% paraformaldehyde, and stained with 0.2% crystal violet. Colonies composed of more than 50 cells were counted. An average surviving fraction was obtained from three parallel experiments.

Surviving Fraction=(Number of colonies formed by cell clones)/(Number of inoculated cells×Inoculation efficiency)

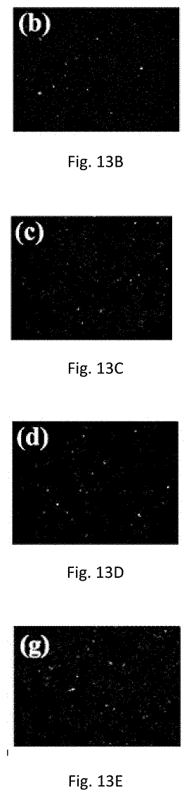

During culture, G-SDS_{6%} showed higher cell attachment and proliferation abilities due to exposure of amino groups, which is beneficial to cell viability. The cells were treated differently (the control group and the G-SDS_{6%} group which was repeated twice), and 8 h later, cell colonies were counted (FIG. 12). The numbers of colonies in the control group and G-SDS_{6%} group are only slightly different, which indicates that the trace amount of surfactant in the collagen polypeptide monolayer has no effect on cell viability. Therefore, the surface of the polypeptide monolayer obtained in the present disclosure has good cytocompatibility, which enables it to be applied to cardiovascular stents.
7. Test of Stability of the Monolayer

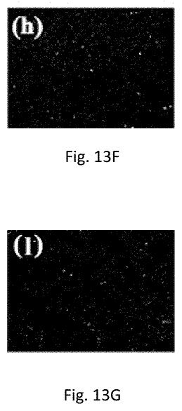

Stability of the collagen polypeptide monolayer was tested by using a DMI3000B inverted fluorescence microscope (Leica, Germany) equipped with a Lecia DFC 450C CCD. After being immersed in normal saline at room temperature for 7 d, the samples were blow-dried with high-purity nitrogen for later use. G-SDS_{6%} was placed in a biochemical incubator at 40° C. for immersion for another 15 d, and blow-dried with high-purity nitrogen for later use. Before observation, it is necessary to turn on a fluorescence module, and the machine was preheated for 15 min and then used. A glass slide was cleaned, the sample to be tested was placed onto the clean glass slide, the glass slide was fixed on an objective table, the height of the objective table was roughly adjusted, then the focus was fine-tuned, the clearest sample details were found with a bright field and observed by using the fluorescence module, the distribution of fluorescent spots was observed under 50× magnification, the magnification was enlarged in sequence to observe the distribution of fluorescent spots, and stability can be analyzed visually by comparison of the distribution of fluorescent spots before and after the immersion of collagen polypeptide monolayer. Results are shown in FIG. 13. After the sample is immersed for 1 week, green fluorescent spots are not decreased. After the sample is placed in the thermostat at 40° C. for 15 d, the distribution of fluorescent spots does not change significantly. Based on the above results, it can be obtained that a relatively stable G-SDS_{6%} monolayer is formed on the surface of Ti, and this stability is attributed to electrostatic and other non-covalent interactions between PEI and collagen polypeptides.

What is claimed is:

1. A polypeptide monolayer with a high surface potential and super-hydrophilicity, characterized in that the polypeptide is composed of polypeptide molecules with a molecular weight of (1.48±0.2)×10⁵ g/mol, a height of the monolayer is 13.8-14.9 nm, the exposure of primary amino groups on the surface of the monolayer is 12-14%, a Zeta potential of the polypeptide monolayer is (−1)-5 mV; and a contact angle of the monolayer is 10±1°.

2. The polypeptide monolayer according to claim 1, characterized in that the polypeptide is collagen polypeptide, and the polypeptide consists of 7.30±0.5% of glycine (Gly); 17.48±0.5% of valine (Vla); 36.97±0.5% of isoleucine (Ile); 13.85±0.5% of leucine (Leu); 2.68±0.5% of tyrosine (Tyr); 1.5±0.5% of phenylalanine (Phe); 4.41±0.5% of lysine (Lys); 0.45±0.5% of histidine (His); 3.45±0.5% of arginine (Arg); 5.96±0.5% of proline (Pro); and 5.95±0.5% of cysteine (Cys).

3. The polypeptide monolayer according to claim 2, characterized in that secondary structures of the polypeptide monolayer comprise 40-51% of α-helix; 10-15% of β-sheet; 2-7% of β-turn; and 31-42% of random coil;
preferably, the secondary structures of the polypeptide monolayer comprise 50.98±0.2% of α-helix; 10.85±0.13% of β-sheet; 6.61±0.07% of β-turn; and 31.56±0.27% of random coil;
or, 40.73±0.1% of α-helix; 14.97±0.13% of β-sheet; 2.55±0.08% of β-turn; and 41.75±0.22% of random coil.

4. The polypeptide monolayer according to claim 1, characterized in that the polypeptide monolayer is composed of close-packed nanoparticles, and the spherical nanoparticles have an average particle size of 60±2 nm.

5. The polypeptide monolayer according to claim 1, characterized in that the exposure of primary amino groups on the surface of the monolayer is 12.47±0.3% or 13.13±0.3%; and the Zeta potential of the polypeptide monolayer is −(0.85±0.1) mV or 4.907±0.1 mV.

6. A composite film containing a polypeptide monolayer, characterized by comprising a polyethyleneimine thin film and the polypeptide monolayer according to claim 1, wherein the polyethyleneimine thin film and the polypeptide monolayer are bound together via ionic bonds, a height of the polyethyleneimine thin film is 0.25-0.38 nm, and a height of the polypeptide monolayer is 13.8-14.9 nm.

7. The composite film according to claim 6, wherein the composite film is prepared by the following steps:
(1) preparing a polypeptide solution at certain temperature, adding sodium dodecyl sulfate (SDS) serving as a surfactant to obtain a polypeptide-SDS mixed solution, and keeping the temperature of the mixed solution, wherein the concentration of SDS in the mixed solution is 3.5-8.32 mmol/L;
(2) grinding and polishing the surface of a titanium sheet, immersing the titanium sheet in a mixed acid solution for treatment, rinsing until the titanium sheet is neutral, blow-drying with nitrogen, and oven-drying;
(3) immersing the oven-dried titanium sheet in an aqueous solution of polyethyleneimine (PEI) for treatment, rinsing with water, blow-drying with nitrogen, and oven-drying to obtain a positively ionized titanium sheet deposited with PEI; and
(4) immersing the positively ionized titanium sheet in the polypeptide-SDS mixed solution obtained at step (1), depositing for 8-12 min, pulling the titanium sheet 20-25 times in deionized water, and blow-drying with high-purity nitrogen to obtain a polypeptide monolayer.

8. The composite film according to claim 7, characterized in that the temperature at step (1) and the temperature during deposition at step (4) are both 50° C.; at step (1), a concentration of collagen polypeptide solution is 4 wt %; the concentration of SDS in the mixed solution is 3.5 mmol/L or 8.32 mmol/L; at step (1), a preparation method of the collagen polypeptide solution comprises the following steps: mixing collagen polypeptides with deionized water, swelling at room temperature for 0.5 h, heating to 50° C., stirring for 2 h until the collagen polypeptides are completely dissolved; and then regulating the pH to 10.00±0.02.

9. The composite film according to claim 7, characterized in that at step (2), after being ground and polished by using metallographical sandpaper, the titanium sheet is ultrasonically washed with deionized water, absolute ethanol, and acetone in sequence for 15 min for each time, blow-dried with high-purity nitrogen, and dried in an oven at 60° C. for 12 h. Further preferably, a grinding and polishing method comprises the following steps: grinding and polishing by using metallographical sandpaper to 800, 1,500, 3,000, 5,000, and 7,000 meshes in sequence;
at step (2), the mixed acid solution is a mixed solution of 30% H₂O₂ and 98% H₂SO₄ in a volume ratio of 1:1, and the treatment time is 1 h; and
at step (3), the titanium sheet is treated with the aqueous solution of PEI for 20-40 min.

10. Application of the polypeptide monolayer according to the composite film according to claim 6 serving as a surface coating material of a cardiovascular stent to treatment of cardiovascular diseases.
